(19) United States
(12) Patent Application Publication
(10) Pub. No.: US 2022/0080200 A1
(43) Pub. Date: Mar. 17, 2022

(54) LEAD DESIGN AND METHODS FOR OPTIMAL LEAD PLACEMENT AND FIELD STEERING

(71) Applicant: SynerFuse, Inc., Minneapolis, MN (US)

(72) Inventors: Gregory F. Molnar, Blaine, MN (US); Nazmi Peyman, Glen Allen, VA (US); Christopher G. Frank, St. Paul, MN (US)

(21) Appl. No.: 17/477,280

(22) Filed: Sep. 16, 2021

Related U.S. Application Data
(60) Provisional application No. 63/079,555, filed on Sep. 17, 2020.

(51) Int. Cl. A61N 1/36; A61N 1/05

(52) U.S. Cl. CPC A61N 1/36071; A61N 1/36125; A61N 1/0551

(57) ABSTRACT

The present invention provides an improved lead design and method for optimal lead placement during a single surgical method for implantation at a spinal treatment site that comprises both targeted vertebral and spinal levels to be treated, wherein the spinal levels comprise at least one dorsal root ganglion. Electrical fields may be generated and shifted in location to optimize stimulation targeting. A spinal treatment procedure is performed generally in combination with implantation of a neuromodulation system that may comprise placement of electrical lead(s) on the at least one dorsal root ganglion, wherein each lead is in operative connection with a pulse generator that may also be implanted during the surgical method. Electrical stimulation may be generated with the pulse generator through the electrical leads to the at least one dorsal root ganglion during and/or after the closure of the identified spinal treatment site.

### CROSS-REFERENCE TO RELATED APPLICATIONS

This application claims the benefit of U.S. provisional patent application No. 63/079555 filed on Sep. 17, 2020 and entitled LEAD DESIGN AND METHODS FOR OPTIMAL LEAD PLACEMENT AND FIELD STEERING, the entire contents of which is incorporated herein by reference.

### STATEMENT REGARDING FEDERALLY SPONSORED RESEARCH OR DEVELOPMENT

Not Applicable.

### BACKGROUND OF THE INVENTION

### Field of the Invention

The invention relates to an improved system and/or method for treating chronic spinal pain comprising a surgical procedure either alone or in combination with a spinal procedure such as vertebral fusion with implantation of a neuromodulation device, wherein the surgical procedure includes lead placement with open physical and visual access to the region of the spine undergoing treatment and/or as a minimally invasive surgical procedure for placement of the lead in the absence of such physical and visual access to the target region.

### Description of the Related Art

Neuromodulation for the treatment of chronic spinal pain is a procedure that has been in use for decades. The procedure is generally prescribed to a patient only after they have gone through a spinal procedure that may involve vertebral fusion in an effort to mitigate and/or correct the supposed source of the pain. However, often such spinal procedures do not resolve the pain issues. After weeks, months and perhaps years of continued chronic pain and pain therapy through medications, including opioids, the patient may finally be prescribed neuromodulation for the treatment of chronic pain after failed back surgery.

The prior art neuromodulation systems include an implantable pulse generator (IPG) and one or more neurostimulation leads having a distal portion having one or more electrodes and a proximal portion for electrically coupling the electrodes to the implantable pulse generator. The lead is implanted via a tunneling method, without direct physical or visual access to the target nerve, such that the electrodes are advanced to a position at or near a target nerve and the implantable pulse generator is implanted in a pocket spaced from the target area.

Existing neuromodulation systems include: the Intellus™ and the Restore Sensor™ systems from Medtronic, PLC; the Spectra™ system from Boston Scientific, Inc; the Senza™ and Omnia™ systems by Nevro, Inc.; the Proclaim™ system by Abbot, Inc.

Problems with the prior art neuromodulation systems include difficulty in achieving satisfactory pain relief due to difficulties with lead placement relative to the nerve target, whether the nerve target is the spinal cord in the case of spinal cord stimulation system or the dorsal root ganglia in the case of dorsal root ganglia stimulation system. Additionally, there is a problem in the prior art of maintaining satisfactory pain relief over time due to lead migration, reduced patient response to a previously efficacious therapy, or due to implantation of the neuromodulation system weeks, months or years after a spinal fixation procedure, or implantation of the neuromodulation system at a different spinal level than the spinal fixation procedure, and other shortcomings that are addressed by the present invention. These problems of the prior art exist both in the case of spinal cord stimulation and dorsal root ganglia stimulation.

Accordingly, it would be highly advantageous to provide a surgical method and system that enables both a spinal procedure and neuromodulation system implantation within a single procedure.

It would be further highly advantageous to enable full physical and visual access to the associated spinal treatment site for placement of the surgical fusion device and the neuromodulation system.

It would be a further advantage to provide a surgical procedure that does not require advancement of an electrical lead through a patient's anatomy to reach the ultimate location of therapeutic efficacy.

It would be a further advantage to provide a surgical procedure and lead design and method of implantation that ensures optimal lead placement.

It would be a further advantage to provide a surgical procedure and lead design and neuromodulation system design that minimizes lead migration over time and/or minimizes loss of therapy over time for a previously efficacious therapy.

It would he a further advantage to provide implantation of the neuromodulation system during the open spinal procedure, wherein the neuromodulation system may generate electrical stimulation during and/or after the surgical procedure.

It would be a further advantage to provide the implanted neuromodulation system as described above and for use in generating electrical stimulation only if the patient experiences pain after the surgical procedure.

It would, alternatively, be advantageous to provide an implantation of the neuromodulation system that would remove the need for the patient to undergo a first spinal fixation surgery, a revision of the first spinal fixation surgery or a subsequent spinal fixation surgery for the treatment of chronic pain

It would further be advantageous to use the neuromodulation system in combination with a laminectomy procedure and/or prevent the need for a laminectomy procedure.

Various embodiments of the present invention address these, inter alia, issues.

### BRIEF SUMMARY OF THE INVENTION

The present invention provides an improved lead design and method for optimal lead placement during a single surgical method for implantation at a spinal treatment site that comprises both targeted vertebral and spinal levels to be treated, wherein the spinal levels comprise at least one dorsal root ganglion. Electrical fields may be generated and shifted in location to optimize stimulation targeting. A spinal treatment procedure is performed generally in combination with implantation of a neuromodulation system that may comprise placement of electrical lead(s) on the at least one dorsal root ganglion, wherein each lead is in operative connection with a pulse generator that may also be implanted during the surgical method. Electrical stimulation may be generated with the pulse generator through the electrical leads to the at least one dorsal root ganglion during and/or after the closure of the identified spinal treatment site.

The Figures and the detailed description which follow more particularly exemplify these and other embodiments of the invention.

### BRIEF DESCRIPTION OF THE SEVERAL VIEWS OF THE DRAWINGS

FIG. 1a illustrates a cutaway view of one embodiment of the present invention.

FIG. 1b illustrates a cutaway view of one embodiment of the present invention.

FIG. 1c illustrates a cutaway view of one embodiment of the present invention.

FIG. 1d illustrates a cutaway view of one embodiment of the present invention.

FIG. 2a illustrates a cutaway view of one embodiment of the present invention.

FIG. 2b illustrates a cutaway view of one embodiment of the present. invention.

FIG. 2c illustrates a cutaway view of one embodiment of the present invention.

FIG. 2d illustrates a cutaway view of one embodiment of the present invention.

FIG. 3a illustrates embodiments of stimulation waveforms and combinations of stimulation waveforms in accordance with the present invention.

FIG. 3b illustrates embodiments of stimulation waveforms and combinations of stimulation waveforms in accordance with the present invention.

FIG. 3c illustrates embodiments of stimulation waveforms and combinations of stimulation waveforms in accordance with the present invention.

FIG. 4a illustrates an embodiment of the present invention with placed leads.

FIG. 4b illustrates a cutaway view of an embodiment of the present invention.

FIG. 5 illustrates another embodiment of the present invention configured for prevention of the onset of leg pain expressed after a spinal fixation procedure.

FIG. 6 illustrates an embodiment of the present invention.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Optimal Lead Placement over the Dorsal Root Ganglia

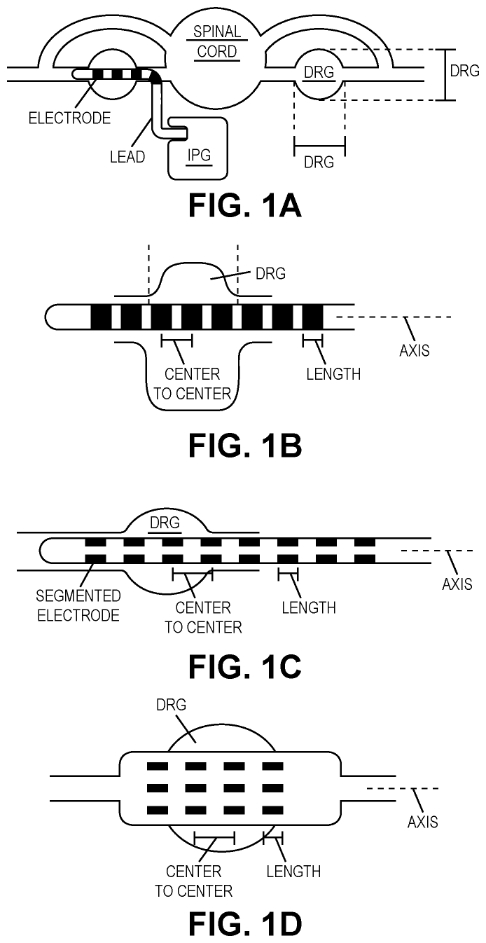

FIGS. 1a-1d illustrate embodiments of a system and method for lead placement and positioning over the target dorsal root ganglia.

In order to describe the embodiments referred to in FIGS. 1a-1d additional anatomical description may be useful. A cutaway of a spinal cord at a first spinal level is shown. The spinal cord is shown having a main body portion. The dorsal root and ventral root are nerve extensions that are spaced from each other, both originating in the main body portion of the spinal cord and extend outwardly therefrom. The dorsal root extends to a dorsal root ganglion having a nodular shape. The dorsal root ganglia has a first side wherein it is connected to the dorsal root and an opposing second side where it is connected to peripheral nerves with an axis of the dorsal root ganglion extending between the first side and second side of the dorsal root ganglion. The ventral root extends from the spinal cord and becomes a spinal nerve that connects to the peripheral nerves on the second side of the dorsal root ganglia.

The dorsal root ganglion, as defined by the nodular shape having a first and second side, is understood to have a length ranging from 2 mm to 10 mm in the human anatomy, and a diameter ranging from 2 mm to 10 mm in the human anatomy. Given this range, one can appreciate that some subjects may even have a dorsal root ganglia with a diameter less than 2 mm or greater than 10 mm. Further, the actual diameter of a particular target dorsal root ganglia will depend upon the patient-specific anatomy and the spinal level of the dorsal root ganglia as each can be a factor in dorsal root ganglia size.

A target contact space is defined by the radial space extending from the axis of the dorsal root ganglia. The target contact space may further be defined by the radial space extending radially outwardly from the periphery of the dorsal root ganglia, in a direction perpendicular to the axis of the dorsal root ganglia.

Turning now to FIG. 1a, a distal portion of a percutaneous lead is shown. The lead having a generally tubular shape with one or more electrodes spaced from each other along a length of an axis defined by the tubular shape of the lead body. Each electrode has an electrode length defined by the distance from a first side and a second side of the electrode along the axis. Each electrode has an electrode diameter as defined by a radial axis extending perpendicular to the elongate axis defined by the tubular body. Each electrode has a contact surface defined by the portion of the lead visibly exposed from the tubular body of the lead.

A placement space is defined by the radial space extending from the periphery of the contact surface, perpendicular to the axis of the lead. The placement space extends along the entire length of the contact space of electrode from the first side to the second side of the electrode.

The lead has a proximal portion for electrically coupling each of the one or more electrodes to an implantable pulse generator. Each of the one or more electrodes is capable of being activated either alone or in combination with other electrodes to selectively deliver an electrical stimulation signal to an anatomical target in proximity to the one or more activated electrodes.

In the embodiments shown in FIGS. 1a-1d, the anatomical target for electrical stimulation is the dorsal root ganglion. The lead 10 is positioned such that at least a portion of the contact surface of at least one electrode 12 is positioned within the target contact space. In some embodiments, the lead 10 is positioned such that the entire contact surface of at least one electrode 12 is positioned within the target contact space. In other embodiments, the lead 10 is positioned with respect to the dorsal root ganglia such that at least a portion of the placement space intersects at least a portion of the target contact area. In yet another embodiment, the lead 10 is positioned and dimensioned such that the entire contact surface of the lead 10 is positioned within the target contact space. In a further embodiment, the lead 10 is positioned and dimensioned such that the entire dorsal root ganglia is located within the placement space.

FIG. 1a shows a top view of a plurality of electrodes 12 being positioned over or along the lead 10 wherein the electrode(s) 12 is/are positioned laterally with respect to the patient anatomy such that the distal portion of the lead 10 extends generally perpendicularly from the spinal cord. in a medial to lateral or lateral to medial relationship to the spinal cord. FIG. 1b shows a top view of a lead 10 with a plurality of electrodes 12 positioned over the dorsal root ganglia. As such, the electrodes 12 may be positioned. in any position and intermediate positions between a lateral and perpendicular placement. Likewise, the relationship between the distal portion of the lead 10 and proximal portion of the lead 10 may exhibit any number of relationships, including but not limited to a proximal portion that extends from the spinal cord to the dorsal root ganglia, a proximal portion that extends from a spinal level above or below the target dorsal root ganglia, a proximal portion that is not axially aligned with the distal portion of the lead such as a medial to lateral approach or a lateral to medial approach of the proximal to distal potions, for example.

FIG. 1c illustrates an alternative embodiment of the lead 10 and method of placement described above with reference to FIGS. la and lb wherein a segmented lead 10 is shown having one or more electrode 12′ locations are defined as being segmented about a radial axis of the lead, such segments may include two or three or more segments and may be concentrically spaced or non-concentrically spaced along the axial length of the lead.

FIG. 1d illustrates an alternative embodiment wherein the lead is a paddle lead,

In one embodiment of the present invention, a lead 10 having two or more electrodes 12 is dimensioned and spaced such that only a single electrode 12 is positionable within the target contact area of the dorsal root. ganglia at a time. In one embodiment, the distance between adjacent electrodes along the length of the lead 10 is such that only a single one electrode 12 is positionable within the target contact space.

In an alternative embodiment of the present invention, the leads 10 may be spaced and dimensioned such that two or more leads 10 are capable of being simultaneously positioned within the target contact space of the dorsal root ganglion such that at least a portion of the contact surface of each of the two or more electrodes 12 is positioned and/or positionable within the target contact space.

One method for placing a lead 10 in accordance with the above embodiments is to provide direct open visual and physical access to the target dorsal root ganglia and directly place the lead, such lead 10 being any of the embodiments described herein or variations thereof, such that at least a portion of the contact. surface of only a single electrode 12 is positioned over the target contact space of the dorsal root ganglia.

Another method of placing the lead 10 in accordance with the present invention is to provide direct open visual and physical access to the target dorsal root ganglia such that the entire length of at least one electrode 12 is positioned within the target contact space defined by the dorsal root, ganglia.

Yet another method is to perform the above lead placement steps at a spinal treatment site in combination with a spinal fixation procedure. In such case, the treatment site is accessed as part of the spinal treatment procedure and the direct visual and physical access to the dorsal root ganglion is provided by virtue of the access to the treatment site created for the spinal treatment procedure. The lead 10 and associated at least one electrode 12 are placed at least partially within the target contact space under direct visual and physical access to at least a portion of the dorsal root ganglion during, after, or in combination with the spinal fixation procedure.

Where the target dorsal root ganglion is at the same spinal level as the spinal fixation then the lead can be placed in accordance with the embodiments of the present invention and lead migration is minimized and/or eliminated due to the spinal fixation procedure resulting in the implantation of fixation rods and screws that prevent movement and bending at the spinal level of the target dorsal root ganglion, and therefore, preventing or minimizing the forces that would otherwise potentially cause lead migration at a spinal level at which spinal fixation had not been performed.

Once the lead 10 is positioned over the target dorsal root ganglion in accordance with the embodiments provided herein, the proximal portion of the lead 10 being electrically coupled to the implantable pulse generator (IPG), the electrode 12 that is positioned over the dorsal root ganglion can be activated by programming of the implantable pulse generator to create an electrical stimulation field around the electrode 12 such as by designating the target electrode as the cathode 16. The anode 14 may then be designated by any conducting member so designated by the user, such as the implantable pulse generator IPG itself being the anode, the fixation rod being designated the anode, the pedicle screw being designated the anode or combinations thereof. Additionally or alternatively, any other additional electrode 12 may be designated as the anode. The neuromodulation electric field is shaped more monopolar/unipolar when the anode is at least two lengths of the target electrode away. The neuromodulation electric field is shaped as a bipolar field when the anode and cathode are less than that distance apart. Thus, in the case where a unipolar field is preferred, the electrode 12 designated as the anode 14, as programmed by the implantable neurostimulator (IPG), should be a distance of at least two lengths of the target electrode away so as to ensure that a generally spherical neurostimulation field is created by the target electrode. Conversely, in the case where a bipolar field is preferred, the electrode designated as the anode, as programmed by the implantable neurostimulator (IPG), should be a distance of less than two electrode lengths away from the target electrode.

As such, electrode 12 size and spacing will determine the neurostimulation field surrounding the dorsal root ganglion. An electrode 12 size of about 0.5 mm to about 10 mm is within the length of electrode contact areas anticipated by the present embodiments. As such, the spacing from one electrode 12 to the next would vary in accordance with the electrode size, as defined by its length.

By way of example, a first 12 electrode having a length of 1 mm may he spaced from a second electrode 12 having a length of 1 mm such that the first and second electrode are spaced from each other such that a center to center distance between the electrodes is less than or equal to 2 mm. For other electrode sizes, lengths, a proportional center-to-center distance would be used in order to provide a bipolar electrical field capability.

Field Shaping and Field Steering

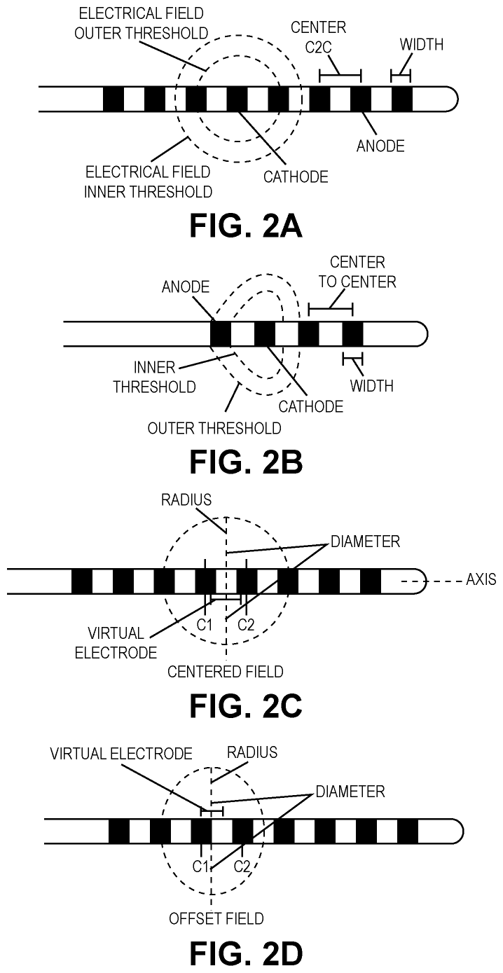

As will be discussed in more detail below with reference to FIGS. 2a-2d, field steering can occur between two adjacent electrodes 12 that are spaced less than two electrode widths of distance apart. In the case where the spherical neurostimulation field of FIGS. 2a, 2c and 2d are preferred either by a patient or implanting physician or by practical and anatomical limitations, there may be at least one intermediate electrode positioned between the cathode electrode and. anode electrode. In the alternative, as shown by way of a non-limiting example in Fib. 2b, a non-spherical electrical field shape may be created wherein an anode may be activated that is within proximity to a cathode that would enable such non-spherical shaping of the electrical field.

The neurostimulation field settings can be programmed by the implantable pulse generator (IPG), and/or an associated patient and/or physician programmer. As shown, the neurostimulation field may be increased or decreased to various levels, the field lines indicating the distance at which a given neurostimulation field meets a minimal threshold to neuromodulate the neurons and/or cells within the defined neurostimulation field. Alternatively, the neurostimulation field can be adjusted in accordance with patient feedback such as alleviation of pain symptoms, presence or absence of paresthesia or other patient or physician articulated criteria. Alternatively, the neurostimulation pattern may be altered such that a particular target area of the dorsal root ganglion undergoes neuromodulation at higher or lower level of neuromodulation activation even where all of such levels of neuromodulation are above a threshold level. As such, the neurostimulation field can be adjusted according to a first level of neuromodulation activation, a second level of neuromodulation activation and additional levels of activation according to patient preference or sensation and/or in consideration of the battery longevity of the implantable pulse generator and/or other considerations alone or in combination.

It may be preferable that certain sub-sections of the dorsal root ganglia are desired to be stimulated and/or to avoid stimulation of certain sub-section of the dorsal root ganglia. For example, it may be desired that the neurostimulation field include a surface of the dorsal root ganglia or a deeper level of the dorsal root ganglia or the peripheral nerve section or the dorsal root section or neither or both depending once again on patient feedback and other considerations.

It may additionally or alternatively be preferable to provide or minimize neurostimulation of the patient anatomy around the dorsal root ganglia. For example, but not intended to be limiting, it may be beneficial to include the spinal root in the neurostimulation field or it may be beneficial to prevent the neurostimulation field from encompassing the spinal root, depending again on patient, physician, anatomical, practical and procedural feedback and limitations.

As shown in FIG. 1c, a segmented electrode would provide a neurostimulation field directed towards the dorsal root ganglia and minimizing, relative to the continuous percutaneous lead of FIGS. 1a and 1b, the directed electrical field facing toward the dorsal root ganglia or other nerve target. Likewise, the paddle lead in FIG. 1d would provide a directed. neurostimulation field with the cathode electrode selection, the anode electrode being positioned at least two electrode lengths away so as not to create a field steering effect.

Field Steering

FIGS. 2a-d illustrate the exemplary use of field steering and/or field shaping in accordance with the various methods, systems and embodiments of the present invention.

FIG. 2a shows an embodiment of the present invention wherein the selected anode and cathode electrode are spaced less than two electrode lengths apart.

FIG. 2b shows in embodiment of the present invention wherein two cathodes and one anode are used to create a non-spheroidal neurostimulation field.

In accordance with the above embodiments shown in FIGS. 2a-d, the neurostimulation field can be located and/or moved based on the positioning and relative ratio of activity (i.e. amplitude) provided to the one or more active anodes and the one or more active cathodes.

Field steering as that term is used herein allows an electric/neurostimulation field to be optimally positioned, shaped, located and/or moved or shifted between adjacent, or non-adjacent, electrodes thus allowing for more programmability with fewer electrodes. Certain embodiments allow the generated electric field to have an initial location along the lead, wherein the initial location comprises flowing current to certain electrodes along the lead that are designated as cathode(s) and as designated anode(s). This initial location of the generated electric/neurostimulation field obviously impacts certain tissue, e.g., the targeted tissue, e.g., DRG or dennatome, and does not impact (or impacts at a lesser stimulation level) other, perhaps non-targeted, tissue. The initial location of the generated electric field may be shifted along the lead to a second location by, inter alia, designating certain electrodes (different in combination that those in the initial location described above) as designated cathode(s) and designated anode(s). Because each electrode along the lead has its own electrical connection with the IPG, the electrodes may be independently selected/designated and energized by the IPG to create a plurality of serial electrical fields, each with a location that may differ from the location of other electrical field locations. As will now be clear, the generated electric/neurostimulation field location may be moved or shifted in a linear, non--linear, regular or random patterned fashion along the lead as the designated cathode(s) and anode(s) are changed.

FIG. 2a shows a single electrode being activated as a cathode, thus creating a spherical electrical stimulation field centered around the activated cathode. The plurality of electrodes along the lead are numbered from 1 (most proximal to IPG) to 8 (most distal from IPG), to help identify certain features in FIGS. 2a, 2c and 2d. The skilled artisan will recognize that 8 electrodes is exemplary, and non-limiting, such that, fewer or greater than 8 electrodes may also be used, each such configuration is within the scope of the present invention.

As discussed above, the IPG may be programmed to select one, or more, of the electrodes along the lead as a cathode and an adjacent, or non-adjacent, electrode as an anode, thereby generating a spherical electrical field. In FIG. 2a, electrode number 4 is the designated cathode 16 and one of either electrodes 3 or 5 is the designated anode. The IPG, or operator, may also select a different pair of electrodes along the lead to effective shift or field steer the electrical field to a new location. This shifting or field steering may move through a plurality of electrodes combinations along the lead and may do so in a linear, non-skipping, configuration. For example, in succession, electrodes 2, 3, 4, 5, 6, 7 may be designated as cathodes wherein one of the electrodes adjacent to the designated electrode may be designated as an anode. Other combinations of electrodes are possible as the skilled artisan will recognize. Alternatively, the designated cathode and related anode pair and generated spherical electrical field, may be designated in a linear, or non-linear pattern, and/or may comprise a random designation of cathode and anode pairs. Optimizing the location(s) of the generated spherical electrical field(s) may comprise using patient feedback and/or Other means. In some cases, a single cathode, e.g., electrode 4 designated as cathode 16 in FIG. 2a may be coupled with two anodes 14, both adjacent designated cathode 16. Thus the electrodes numbered as 3 and 5 in FIG. 2a may serve as designated anodes 14 and the electrode numbered as 4 may serve as the designated cathode 16. Again, this configuration may be shifted or field steered along the lead in a linear or non-linear pattern and/or randomly.

FIG. 2b shows a lead having a plurality of electrodes, e.g., 4, each having a length and wherein the center-to-center distance between the activated designated cathodes 16 (electrodes numbered 2 and 3 in FIG. 2b) and the single activated designated anode electrode 14 (electrode numbered 2 in FIG. 2b) is less than two electrode lengths. This results in a non-spherical, oblong, or tear-drop shape having a greater electrical field radius near the cathode and a lesser diameter near the anode. FIG. 2b illustrates an exemplary and non-limiting number of electrodes, numbered from 1 (most proximal to IPG) to 4 (most distal from IPG). As described in connection with FIG. 2a, this configuration may be shifted or field steered along the lead by changing the position of the designated cathodes 16 and the related anode 14, to move the generated electrical field to a desired location(s) and in a desired pattern(s) which may include linear, non-linear and/or random movement.

FIGS. 2c and 2d are illustrative of field steering that may be achieved by alternating the amplitude of adjacent or nearby energy-effecting electrodes. As such, the radial center of the electrical field can be shifted from centering over a first electrode or a second electrode to a predetermined position between the two electrodes. The central portion of the electrical field effectively acting as a virtual electrode at the resulting center of the electrical field.

In FIG. 2c, the virtual electrode is positioned between the first and second cathodes 16 (numbered as electrodes 4 and 5 on the lead 10) with one or both of adjacent electrodes (numbered as 3 and 6 on the lead 10) may be designated as anode(s) 14. C1 and C2 designate the respective centers for electrodes numbered as 4 and 5 on lead 10 in FIG. 2c. As described above in connection with FIGS. 2a and 2b, the designated cathode electrodes 16 and anode electrode(s) 14 may be shifted along the lead 10 to field steer the generated electrical field to a desired location and in a desired pattern, frequency, etc.

In FIG. 2d, the virtual electrode is centered at about edge of a first cathode 16 (numbered as electrode 3 on the lead 10) and away from the second cathode 16 (numbered as electrode 4 on the lead 10). As in FIG. 2c, C1 and C2 designate the respective centers for electrodes numbered as 4 and 5 on lead 10 in FIG. 2d. Electrical field steering allows for the virtual electrode to be centered anywhere between the edges of adjacent electrodes, i.e., in the region between electrodes numbered as 3 and 4 on the lead, and alternatively may extend further to the center of the first or the second electrode in such case where only a single electrode is active as a cathode. Such field steering allows for optimal placement of the electrical stimulation field with respect to the target nerve tissue without the requiring specific placement of a single electrode for optimal therapeutic effect. It will be appreciated that applying less (non-zero) voltage and/or current to one of the designated cathode electrodes 16 will also work to shift the virtual electrode toward the designated cathode electrode 16 to which lower voltage and/or current is applied. In this manner, it is possible to shift the virtual electrode (and move the resulting generated electrical field) to infinite positions between and/or along the two adjacent cathodes 16.

The shape and size of the neurostimulation field can be steered according to the selection of one or more adjacent cathodes or cathodes within a field-effecting distance from one another. The selection of the ratio of energy (or amplitude) delivered to each of the one or more anodes and/or cathodes is selected in order to create a predetermined neurostimulation field location and/or shape in order to achieve a predetermined functional, clinical or performance criteria and/or neurostimulation field shape. By way of example, the use of two cathodes for field steering and/or field shaping may also result in a greater radial field of electrical stimulation from the center of the virtual electrode position along the axial length of the lead as compared to a single electrode.

In use, the implantable pulse generator is programmed via a physician and/or patient programmer to active certain electrodes of the lead as cathode and/or anode in accordance with predefined outcome criteria such as pain relief, battery longevity and other considerations either alone or in combination.

Novel Wave Forms

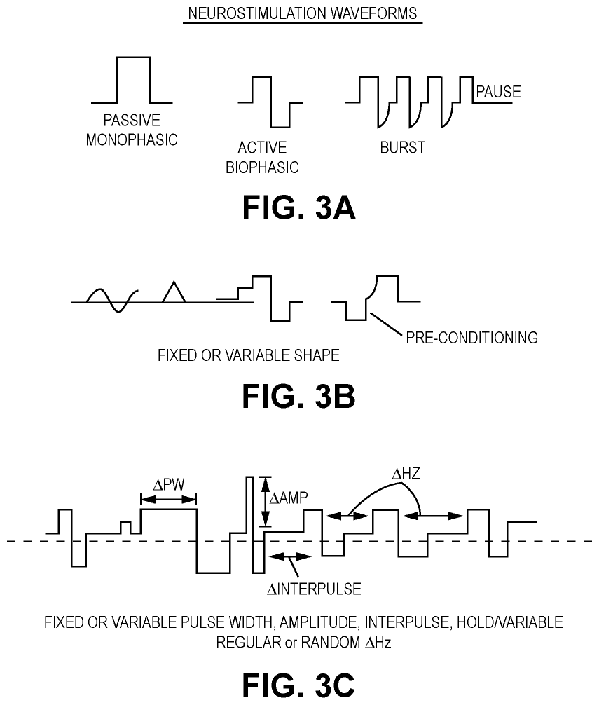

FIGS. 3a-3e illustrate embodiments of stimulation waveforms and combinations of stimulation waveforms in accordance with the present invention.

As discussed above with reference to FIGS. 1a-1d, 1d and 2a-2d, the neuromodulation field is defined by the electrode position, spacing, designation as anode or cathode, and ratio of activation.

Once such neurostimulation field is determined based on patient feedback and other considerations, the wave form can be programmed into the implantable pulse generator to generate a waveform in accordance with patient, physician, functional and practical considerations.

The waveform may take any of the waveforms shown in FIG. 3 and/or may include variations and/or combinations thereof. A particular waveform may result in the desired patient and performance outcome in some embodiments. In other embodiments, the desired patient and performance outcome may be sustained only by variations in the waveform such as varying from a first waveform to a second wave form. And, after a period of time, the desired patient and performance outcomes may be sustained, or improved or minimally diminished by again varying from a current waveform to a new waveform.

Waveforms may be delivered from a library of different waveforms, such as a tonic, burst, high frequency, low frequency, high amplitude, low amplitude, phase shifting, phase locking, phase changing waveform.

Alternatively, the waveforms may be delivered by altering various current waveform parameters without having a predetermined second waveform as a basis for altering the current waveform. In such case, there may be a window of acceptable phases, amplitudes, time periods and/or other parameters within which a new waveform must conform but need not be a predetermine or previously designed waveform, simply a waveform that is sufficiently different from the current waveform in order to provide a therapeutic effect, for example, or to minimize diminution of the therapeutic effect of the current waveform after a period of time.

By way of example, a first waveform may be programmed to be delivered to a target stimulation site for a first predetermined amount of time and then a second waveform may be programmed to be delivered to the target stimulation site for a second predetermined amount of time. Each of the waveforms may be fixed in terms of amplitude and duration or variable in amplitude and duration, but in either case are differentiated from each other such that the neurostimulation waveform at the first period of time is measurably different than the second neurostimulation waveform at the second period of time. Likewise, first and second periods of time may be of the same or differing in length.

The alternative cycle between one waveform and another may be repeated, or a series of non-repeating waveforms and repeating and/or non-repeating time periods may be utilized in accordance with the use and design of the neurostimulation system of the present invention.

The various potential waveforms and/or combinations of waveforms and waveform variables are illustrated in FIGS. 3a-3e. One adjustable variable of the waveform is amplitude, for delivering electrical stimulation as measured in V or mA. A constant current or a constant voltage waveform may be used depending on preference. Pulse width may be another adjustable variable, measurable in microseconds of duration may be typical here. Yet another adjustable variable is frequency, measurable and controllable in Hz.

These and additional control variables may be used to create predetermined waveforms, novel waveforms or combinations of a plurality of predetermined waveforms, or combinations of a plurality of novel waveforms, or mixed combinations of predetermined and novel variable and/or random waveforms.

Waveforms may have a predetermined fixed, repeating, non-repeating, random, non-random form including but not limited to: waveform shape such square, non-square rounded, saw tooth, sloping leading edge, sloping trailing edge, variations or fixed peak, variations or fixed adaptations, may utilized pre-condition pulses, hyperpolarize to make more excitable or depolarize to make less excitable. From a patient point of view, waveforms may be predetermined and/or randomized such that a neuron or group of neurons are or are not recruited via neurostimulation, are or not made more or less excitable to make it more or less likely to fire when modulated, do or do not induce paresthesia and the levels of effect of each of these on a. patient perception of paresthesia, pain, or other patient attributes.

The waveforms may then be titrated to patient perception, for example, providing sub threshold stimulation but resulting in pain relief or paresthesia.

Sensing plus Stimulation (Sensing, Analyzing, Copying and then Stimulating)

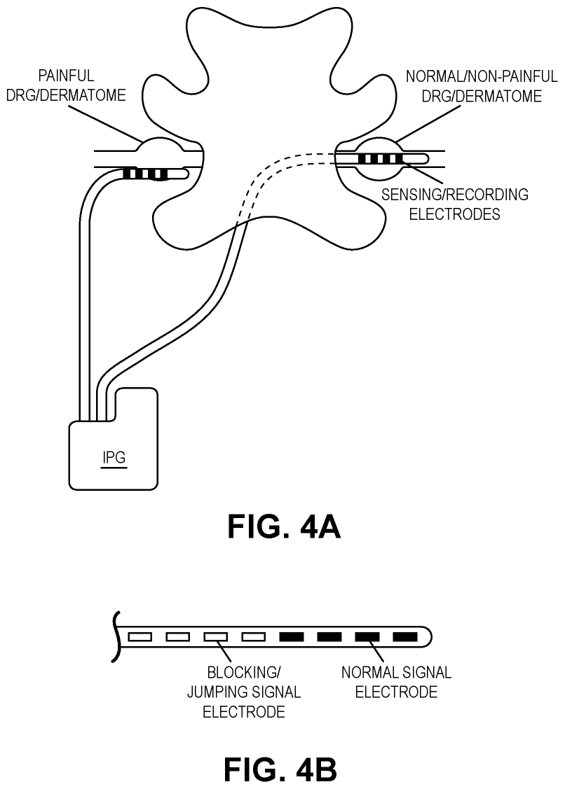

In yet another embodiment of the present invention, what is provided in FIG. 4 is a lead having one or more electrodes at a distal portion thereof wherein the electrodes have a sensing capability and an electrical stimulation capability.

As shown in FIG. 4a, a first lead 10 with a first set of sensing and stimulating electrodes is shown at a first dorsal root ganglia and a second lead 10′ with a second set of sensing 20 and stimulating 22 electrode(s) is shown at a second dorsal root ganglia. The sensing electrode(s) 20 may be placed at a dorsal root ganglion or nerve structure that is not involved in the generation and/or transmission of pain signal and on the ipsilateral or contra-lateral side of the patient's pain. The sensing electrode(s) 20 may sense the normal signals (non-painful signals) and relay the sensed data to the pulse generator/analyzer (IPG). These signals may be analyzed, copied and in some cases modified by the IPG to generate an electrical stimulation pattern, mimicking a normal, healthy signal pattern, intensity and frequency.

This generated stimulation pattern may then be used to stimulate the painful (pathologic) dorsal root ganglion and/or nerve or nervous system structure. This signal would then be transmitted to the higher nervous system structures (higher neuronal structures, brain structures, etc. The rational for this being that the higher structures in the spinal cord and/or brain would not receive the signals and messages from the lower structures (more distal or peripheral structures) indicating pain. They would receive messages and signals that indicate no pain, much like being sent to the higher neuronal structures from the other (non-painful) side.

In another embodiment, illustrated in FIG. 4b, the lead may comprise two parts. The first part (distal and more peripheral part) of the lead may transmit and apply jamming and blocking signals from the IPG, so the painful signals would not reach the more central nerve structures and the second part of the lead (proximal part and the part closer to the spinal cord and central nervous system structures) would receive the non-painful signals that have been sensed, analyzed, copied and possibly modified from the non-painful and healthy ipsilateral or contralateral dorsal root ganglia, nerves or other central or peripheral nervous system structures. In a way, the pain signals will be blocked and replaced by non-painful and normal patterns of messages (the good vibrations).

Any of the one or more electrodes may be programmed to act either as a sensing 20 or as a stimulating 22 electrode by the IPG or the operator. Alternatively, a predetermined one or more electrodes may be sensing only electrodes while a predetermined one or more set of electrodes may be stimulation only electrodes.

Returning to FIG. 4, the first lead 10 may be used for sensing and the second lead 10′ may used for delivering of a stimulation pattern based on the sensing of the first lead. By way of example, if the second lead 10′ is positioned at a dorsal root ganglia associated with pain in the corresponding dermatome and the first lead 10 is positioned at a dorsal root ganglia that is not associated with pain in the corresponding dermatome, the electrical signals that are sensed by the second lead's electrodes 20 are then used to define a waveform pattern that is then delivered to the second lead 10′, such waveform pattern not being based on a predetermined waveform pattern, but instead based upon biologically sensed electrical signals which may vary from one sensing time frame to another and may or may not be a waveform that has previously been used.

In one embodiment, the sensing time frame may be continuous wherein an electrical stimulation pattern is sensed by sensing electrodes 20 of the first lead 10 and then a corresponding electrical stimulation pattern is delivered to the stimulating electrodes 22 of the second electrode 10′ in an essentially continuous fast-following fashion.

In another embodiment, a sensing window is used, wherein a predetermined time of sensed electrical information is received by sensing electrodes 20 of the first electrode 10 and such pattern is then delivered in a repeating fashion to the stimulating electrodes 22 of the second lead 10′ for either a predetermined period of time at which time the process repeats with a new sensing time frame for new stimulation pattern creation.

In yet another embodiment, first and second leads 10, 10′ are placed on separate dorsal root ganglia. Rather than relying on sensing, the first lead 10 while placed on a healthy dorsal root ganglia will stimulate the health dorsal root ganglia in order to reduce the sensation of pain in the dorsal root ganglia that is associated with pain. Without being bound by theory, the intention is to provide a novel signal to the patient which distracts from the pain signals at the second target anatomical zone.

In yet another embodiment, the same lead 10 or 10′ may perform both a sensing function and an electrical stimulation function. Once the sensing function has been performed for a predetermined time frame, a “pain signal waveform” is sensed and then an electrical stimulation is delivered out of phase to the pain signal waveform in order to cancel out the pain signal waveform and therefore reduce the sensation of pain sensed by the patient.

In a further embodiment, either alone or in combination with the above, the lead is implanted at a dorsal root ganglion where a spinal fixation device has been implanted.

In another alternative embodiment of a sensing and neurostimulation system, a sensing of the electrical activity of the brain may be utilized as an input to the sensing-based programming of a neurostimulation output. The brain sensing may be a biomarker that allows for the titration of the neurostimulation signal, and/or the brain sensing can be used to determine a biomarker for use in optimizing the neurostimulation signal. The signal may be titrated based on patient sensation in order to either identify a normal biomarker, abnormal biomarker, ideal biomarker, or combinations thereof. The biomarker allows for sensing of a patient signal that is titrated to a patient sensation such as pain, such that sensing of the biomarker allows for closed-loop or open-loop programming of the neuromodulation parameters and electrode or virtual electrode position, waveform and other attributes in order to provide patient therapy.

Leg Pain Expressed After Completion of a Spinal Fixation Procedure

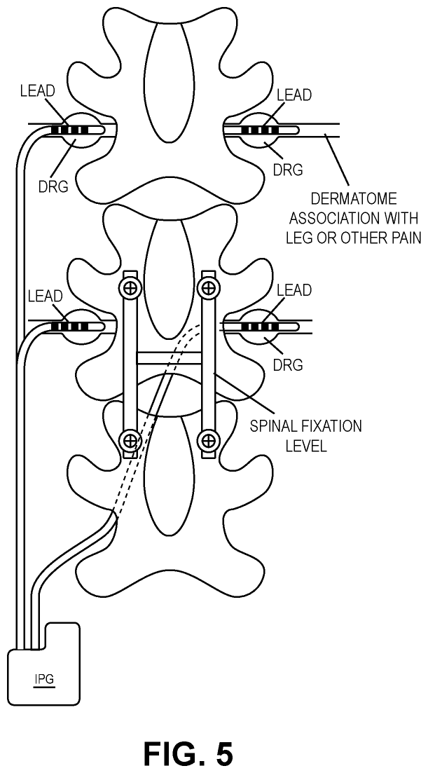

FIG. 5 illustrates a system and method for prevention of the onset of radicular pain, including but not limited to leg pain expressed after a spinal fixation procedure. A first set of leads are positioned at the same spinal level as the spinal fixation device for treating the dermatome associated with patient pain. A second set of leads are placed at the dermatome associated with leg pain. Without being bound by theory, in many cases after having a spinal fixation procedure for treatment of back pain, a patient that previously exhibited no leg pain will begin to exhibit radicular/leg pain. This may be caused by the fact that the nerve pressure on the leg dermatome has been altered in response to the fixation procedure in that the back pain was masking the leg pain or that the change, whether anatomically prescribed or not, causes a pain response. Irrespective of theory, a patient exhibiting radicular/leg pain post spinal fixation procedure can also be treated by the first set of leads at the fusion level or by placement of the additional set of one or more leads at the dorsal root ganglia at the spinal level associated with the radicular leg pain.

Therapies Utilizing Cross-Talking between Different Levels of Dorsal Root Ganglia

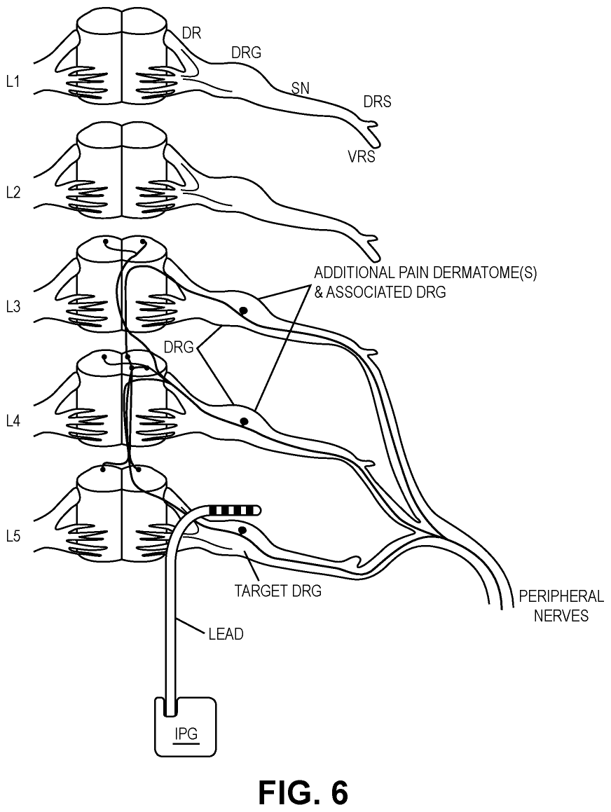

FIG. 6 illustrates another embodiment of the present invention utilizing cross-talk between sensory dermatomes wherein a lead is implanted at a first dorsal root ganglia associated with a first dermatome and a neuromodulation therapy is provided to treat pain associated with a second dermatome. A set of at least one leads are positioned at the dorsal root ganglia associated with the first dermatome at a first spinal level. The patient may be experiencing pain at the dermatome associated with the first spinal level. The patient may also, or exclusively, be experiencing pain at a second dermatome associated with a second spinal level and corresponding second dorsal root ganglia. In such case, the electrical stimulation patterns used to electrically stimulate the first dorsal root ganglion at the first spinal level can be prescribed for the treatment of pain associated with the dermatome of second spinal level. Without being bound by theory, each dorsal root ganglia is generally associated with one dermatome but through the interleaving network of neural tissue is able to exhibit cross-talk between dermatomes and therefore the neurostimulation of a first dorsal root ganglia associated with a first dermatome may be modified and/or programmed to alleviate pain sensations associated with a second dermatome and corresponding second spinal level and second dorsal root ganglia. The first and second dermatomes and associated spinal levels and dorsal root ganglion may be adjacent levels or may be separated by one or more spinal levels therebetween in accordance with the present invention. By way of non-limiting example, the lead may be positioned at the first dorsal root ganglion associated with spinal level L4. The implantable pulse generator may be programmed to deliver an electrical stimulation program to the first dorsal root ganglion, associated with a first dermatome, via the electrodes such that a pain sensation associated with a second dermatome and corresponding second dorsal root ganglion associated with a spinal level L3 is alleviated. Alternatively, such electrical stimulation program delivered to the first dorsal root ganglion may be delivered in order to alleviate a pain sensation associated with a second dermatome, and corresponding dorsal root ganglion associated with a spinal level L4. Similarly, the neuromodulation system of the implantable pulse generator may be programmed to alleviate a pain sensation associated with multiple dermatomes, such as those associated with level L3 and L5 simultaneously. Alternatively, such use of cross-talking between dermatomes may be utilized to alleviate a pain or other sensation associated with a dermatome that is more than one spinal level from the location of the lead and/or dorsal root ganglion of the neuromodulation system.

Further, reference is made to the following United States Patent References, the entire contents of which are incorporated herein by reference:

U.S. patent application Ser. No. 16/519,320 filed on Jul. 23, 2019 entitled METHOD FOR IMPLANTING A NEUROMODULATION SYSTEM AT A SPINAL TREATMENT SITE;

U.S. patent application Ser. No. 16/665,525 filed on Oct. 28, 2019 entitled SYSTEMS, DEVICES AND METHODS FOR IMPLANTABLE NEUROMODULATION STEMULATION; and.

U.S. patent application Ser. No. 16/409,616 filed on May 10, 2019 entitled SYSTEM, DEVICES, AND METHODS COMBINING SPINAL STABILIZATION AND NEUROMODULATION.

The various embodiments described herein can be used as a system or method either alone or in combination with the various elements, features and methods of other embodiments and/or modifications thereof, in accordance with the spirit of the present invention. By way of example, the embodiments of a lead and implantable pulse generated may be implanted individually at a spinal treatment site either alone or in combination with a spinal fixation device. Likewise the lead and implantable pulse generator may exhibit any of the various functions and methods described herein either alone or in combination with a spinal fixation device and either at the same spinal level as a spinal fixation device or at a different spinal level than the spinal fixation device.

The descriptions of the embodiments and their applications as set forth herein should be construed as illustrative, and are not intended to limit the scope of the disclosure. Features of various embodiments may be combined with other embodiments and/or features thereof within the metes and bounds of the disclosure. Upon study of this disclosure, variations and modifications of the embodiments disclosed herein are possible and practical alternatives to and equivalents of the various elements of the embodiments will be understood by and become apparent to those of ordinary skill in the art. Such variations and modifications of the embodiments disclosed herein may be made without departing from the scope and spirit of the invention. Therefore, all alternatives, variations, modifications, etc., as may become to one of ordinary skill in the art are considered as being within the metes and bounds of the instant disclosure. Atty.

What is claimed is:

1. A system for creating a plurality of targeted electrical fields at a plurality of targeted portions of a dorsal root ganglion (“DRG”) in a patient, comprising:
a lead having a distal portion with a plurality of electrodes disposed along the lead;
an implantable pulse generator (“IPG”) in operative electrical communication with each electrode in the plurality of electrodes, wherein one or more of the electrodes comprise a first designated cathode and one or more of the remaining electrodes comprise a first designated anode, the IPG further configured to flow current through the designated electrodes to generate an electrical field at a first location,
wherein the IPG is further configured to designate a second designated cathode comprising one or more electrodes,
wherein the one or more electrodes comprising the second designated cathode are different from those comprising the first designated cathode, and
wherein the IPG is further configured to flow current through the designated electrodes to generate an electrical field at a second location.

2. The system of claim 1, wherein the IPG further configured to designate a second designated anode comprising one or more electrodes, wherein the one or more electrodes comprising the second designated anode are different from those comprising the first designated anode, the IPG further configured to flow current through the designated electrodes to generate an electrical field at a third location.

3. The system of claim 1, wherein the IPO is configured to designate a plurality of designated anodes and designated cathodes, the IPG further configured to flow current through a programmed succession of the designated anodes and designated cathodes to generate electrical fields at a plurality of locations.

4. The system of claim 3, wherein the programmed succession is further configured to generate electrical fields in a pattern.

5. The system of claim 1, wherein patient-provided sensation feedback is used to configure the IPG to produce generated electrical fields with one or more selectable parameters selected from the group consisting of: current flow, time period, frequency, shape and location of the generated electrical field.

6. The system of claim 3, wherein the IPG is further configured to increase or decrease the current level for one or more of the generated electrical fields.

7. The system of claim 3, wherein the IPG is further configured to maintain the generate electrical fields for a period of time, wherein the IPG is configured to increase or decrease the period of time for one or more of the generated electrical fields.

8. The system of claim 3, wherein at least some of the designated anodes and designated cathodes are selected to generate a spherical electrical field.

9. The system of claim 3, wherein at least some of the designated anodes and designated cathodes are selected to generate a non-spherical electrical field.

10. The system of claim 3, wherein the IPG is configured to provide current flow to the designated electrodes in the form of one or more waveforms selected from the group consisting of: high frequency, tonic, burst, tonic, hi frequency, low frequency, amp modulation, and phase changing/locking options.

11. The system of claim 3, wherein the locations of the generated electrical field target stimulation to certain portions of the DRG.

12. The system of claim 3, wherein the locations of the generated electrical field avoid provision of stimulation to certain portions of the DRG.

13. The system of claim 1, wherein the first and second designated cathodes are spaced apart and configured to create a virtual electrode therebetween when the electrical field is generated at the second location.

14. The system of claim 1, wherein the virtual electrode comprises a location that is substantially centered between the first and the second designated cathodes, and wherein the generated electrical field is symmetrical with respect to the first and second designated cathodes.

15. The system of claim 14, wherein the IPG is configured to cause current to flow to the first designated cathode and the second designated cathode, wherein the current level received by the first designated cathode is less than the current received by the second designated cathode, and wherein the virtual electrode is thereby configured to shift location toward the first designated electrode, wherein the generated electrical field is off set from, and is asymmetrical with respect to, the first and second cathodes.

16. A system for creating a plurality of targeted electrical fields at a plurality of targeted portions of a dorsal root ganglion (“DRG”) comprising:
a lead having a distal portion with a plurality of electrodes disposed along the lead;
an implantable pulse generator (“IPG”) in operative electrical communication with each electrode in the plurality of electrodes,
wherein one or more of the electrodes comprise a first designated cathode and one or more of the remaining electrodes comprise a first designated anode, the IPG further configured to flow current through the designated electrodes to generate an electrical field at a first location,
wherein the IPG is further configured to designate a second designated anode comprising one or more electrodes,
wherein the one or more electrodes comprising the second designated anode are different from those comprising the first designated anode, and
wherein the IPG is further configured to flow current through the designated electrodes to generate an electrical field at a second location.

17. The system of claim 16, wherein the IPG is further configured to designate a second designated cathode comprising one or more electrodes, wherein the one or more electrodes comprising the second designated cathode are different from those comprising the first designated cathode, the IPG further configured to flow current through the designated electrodes to generate an electrical field at a third location.

18. The system of claim 16, wherein patient-provided sensation feedback is used to configure the IPG to produce generated electrical fields with one or more selectable parameters selected from the group consisting of: current flow, time period, frequency, shape and location of the generated electrical field.

19. The system of claim 16, wherein the IPG is further configured to increase or decrease the current level for one or more of the generated electrical fields.

20. The system of claim 16, wherein the IPG is further configured to maintain the generated electrical fields for a period of time, wherein the IPG is configured to increase or decrease the period of time for one or more of the generated electrical fields.

21. The system of claim 17, wherein the designated anodes and designated cathodes are selected to generate a spherical electrical field.

22. The system of claim 17, wherein the designated anodes and designated cathodes are selected to generate a non-spherical electrical field.

23. The system of claim 16, wherein the locations of the generated electrical field target stimulation to certain portions of the DRG.

24. The system of claim 17, wherein the locations of the generated electrical field avoid provision of stimulation to certain portions of the DRG.

25. The system of claim 17, wherein the first and second designated cathodes are spaced apart and configured to create a virtual electrode therebetween when the electrical field is generated at the second location.

26. The system of claim 17, wherein the virtual electrode comprises a location that is substantially centered between the first and the second designated cathodes, and wherein the generated electrical field is symmetrical with respect to the first and second designated cathodes.

27. The system of claim 26, wherein the IPG is configured to cause current to flow to the first designated cathode and the second designated cathode, wherein the current level received by the first designated cathode is less than the current received by the second designated cathode, and wherein the virtual electrode is thereby configured to shift location toward the first designated electrode, wherein the generated electrical field is off set from, and is asymmetrical with respect to, the first and second cathodes.
